(19) United States
(12) Patent Application Publication
(10) Pub. No.: US 2020/0353119 A1
(43) Pub. Date: Nov. 12, 2020

(54) AIR PURIFIER AND AIR PURIFICATION METHOD

(71) Applicant: ALPS ALPINE CO., LTD., Tokyo (JP)

(72) Inventor: Hiroyuki HEBIGUCHI, Miyagi (JP)

(21) Appl. No.: 16/940,720

(22) Filed: Jul. 28, 2020

Related U.S. Application Data
(63) Continuation of application No. PCT/JP2018/043976, filed on Nov. 29, 2018.

(30) Foreign Application Priority Data
Feb. 2, 2018 (JP) 2018-017345

(51) Int. Cl. A61L 9/12; A61L 9/04

(52) U.S. Cl. CPC A61L 9/12; A61L 9/046; A61L 2101/06; A61L 2209/212; A61L 2209/111

(57) ABSTRACT

An air purifier includes a purifier, a circulator, a concentration sensor, and a controller. The purifier is configured to remove an odor component from air in a space, and includes a purifying substance generator configured to generate a purifying substance that removes the odor component. The circulator is configured to draw the air in the space into the purifier, and discharge the air purified by the purifier into the space. The concentration sensor is configured to detect the concentration of the purifying substance in the air. The controller is configured to control the amount of the purifying substance generated by the purifying substance generator, in accordance with the concentration of the purifying substance. The controller adjusts the amount of the purifying substance in response to determining that the increase rate in the concentration of the purifying substance per unit time is greater than a predetermined rate.

### CROSS-REFERENCE TO RELATED APPLICATIONS

This application is a continuation of International Application No. PCT/JP2018/043976, filed on Nov. 29, 2018 and designating the U.S., which claims priority to Japanese Patent Application No. 2018-017345 filed on Feb. 2, 2018. The contents of these applications are incorporated herein by reference in their entirety.

### BACKGROUND OF THE INVENTION

### 1. Field of the Invention

The disclosures herein relate to an air purifier and an air purification method.

### 2. Description of the Related Art

Air purifiers are known that purify the air in the interior of a vehicle or in the room of a building by using an oxidizer, such as ozone or hypochlorous acid, to remove (deodorize) or sterilize odor components such as malodorous substances and volatile organic compounds from the air.

As such an air purifier, a ventilation/deodorization device for vehicles is described (Patent Document 1, for example). The ventilation/deodorization device includes a fan, an ozone generator, a switching damper, a control unit, and an odor sensor. In the related-art ventilation/deodorization device, the control unit controls the fan, the ozone generator, and the switching damper, and switches between three modes, a ventilation mode, a normal purification mode, and an ozone fumigation mode, in accordance with the odor concentration in the vehicle detected by the odor sensor. In order to remove a strong unpleasant odor from the vehicle, the ozone fumigation mode is selected, and high concentrations of ozone exceeding a criterion value are generated for a predetermined period of time. As a result, the strong unpleasant odor is efficiently removed from the vehicle.

In general, the odor sensor is a semiconductor sensor that uses a semiconductor device including an oxide semiconductor or an organic semiconductor in most cases. In the semiconductor sensor, the resistance of the semiconductor device changes when oxygen absorbed on the negatively charged surface of the semiconductor device reacts (surface reaction) with odor components and is desorbed from the surface. The semiconductor sensor utilizes the above-described characteristics to measure the concentration of odor components in air.

However, in the case of the semiconductor sensor, if ozone generated for deodorization is present in a vehicle, oxygen ions adsorbed on the surface of the semiconductor device would be increased, thus decreasing the performance of the odor sensor measuring odor components. Therefore, even if odor components remain in the interior of a vehicle or a building, the odor sensor may measure a lower concentration of odor components and the operation of the ozone generator may end prematurely.

Conversely, when no odor sensor is used, the ozone generator needs to be turned on or off after the lapse of a predetermined period of time. Therefore, there may be a possibility that odor components may fail to be completely removed or the ozone generator may be excessively operated.

Therefore, the amount of a purifying substance such as ozone or hypochlorous acid for removing odor components from air needs to be appropriately controlled.

### RELATED-ART DOCUMENTS

### Patent Documents

[Patent Document 1] Japanese Laid-Open Patent Publication No. 10-151941

### SUMMARY OF THE INVENTION

It is a general object of the described embodiments to provide an air purifier that adjusts the generation of a purifying substance at an appropriate timing.

According to an embodiment, an air purifier includes a purifier, a circulator, a purifying substance concentration sensor, and a controller. The purifier is configured to remove an odor component from air in a space, and includes a purifying substance generator configured to generate a purifying substance that removes the odor component. The circulator is configured to draw the air in the space into the purifier, and discharge the air purified by the purifier into the space. The purifying substance concentration sensor is configured to detect the concentration of the purifying substance in the air. The controller is configured to control the amount of the purifying substance generated by the purifying substance generator, in accordance with the concentration of the purifying substance. The controller adjusts the amount of the purifying substance generated by the purifying substance generator by controlling the purifying substance generator, in response to determining that the increase rate in the concentration of the purifying substance per unit time is greater than a predetermined rate.

### BRIEF DESCRIPTION OF THE DRAWINGS

Other objects and further features of the present invention will be apparent from the following detailed description when read in conjunction with the accompanying drawings, in which:

FIG. 1 is a diagram illustrating a vehicle in which an air purifier according to an embodiment is installed;

FIG. 2 is a diagram illustrating an example of a controller;

FIG. 3 is a flowchart illustrating an air purification method;

FIG. 4 is a graph illustrating an example of the relationship between the time and the concentration of ozone; and

FIG. 5 is a diagram illustrating an example of the relationship between a unit time and the amount of increase in the concentration of ozone.

### DESCRIPTION OF THE EMBODIMENTS

According to an embodiment, it is possible to provide an air purifier that adjusts the generation of a purifying substance at an appropriate timing.

In the following, embodiments of the present invention will be described with reference to the accompanying drawings. In order to facilitate understanding of the description, the same elements are denoted by the same reference numerals in the drawings, and a duplicate description thereof will not be provided. Note that the constituent elements illustrated in the drawings may not be to scale.

### <Air Purifier>

An air purifier according to an embodiment of the present invention will be described. The present embodiment describes an example in which the air purifier purifies air in the interior space (space) of a vehicle by removing odor components from the air. The odor components refer to malodorous substances and volatile organic compounds. Examples of odors include cigarette odor, pet odor, human body odor, and body odor of the elderly. Further, in the present embodiment, the purifying substance used to purify odor components is ozone.

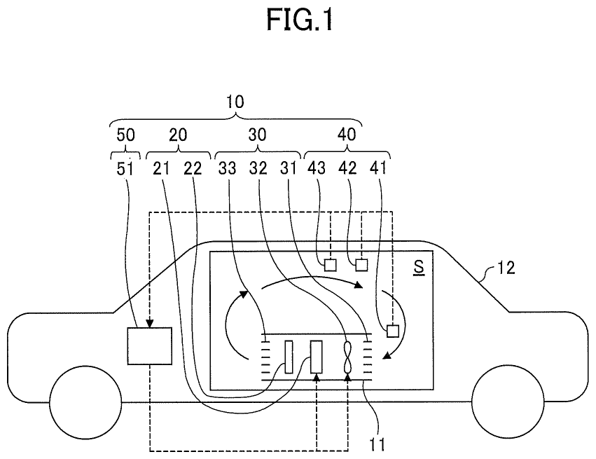

FIG. 1 is a diagram illustrating a vehicle in which an air purifier according to an embodiment is installed. FIG. 1 illustrates a state in which the vehicle is stopped and there is no human or pet (animal) in the vehicle.

As illustrated in FIG. 1, an air purifier 10 includes a purifier 20, a circulator 30, a sensor 40, and a controller 50. The purifier 20, the circulator 30, and the sensor 40 are provided in the interior space (space) S of a vehicle 12, and a main body of the controller 50 is provided outside the space S and inside the vehicle 12. The purifier 20 and the circulator 30 are accommodated in a main body 11 of the air purifier 10.

The main body 11 includes a casing having an interior space in which the purifier 20 and the circulator 30 are accommodated. The interior space has an air passage through which air flows in a predetermined direction. For example, the casing of the main body 11 may have a cylindrical shape or a rectangular shape.

### (Purifier)

The purifier 20 purifies air in the space S, and includes a purifying substance generator 21 and a dust filter 22. The purifying substance generator 21 and the dust filter 22 are arranged in this order in the air flow direction.

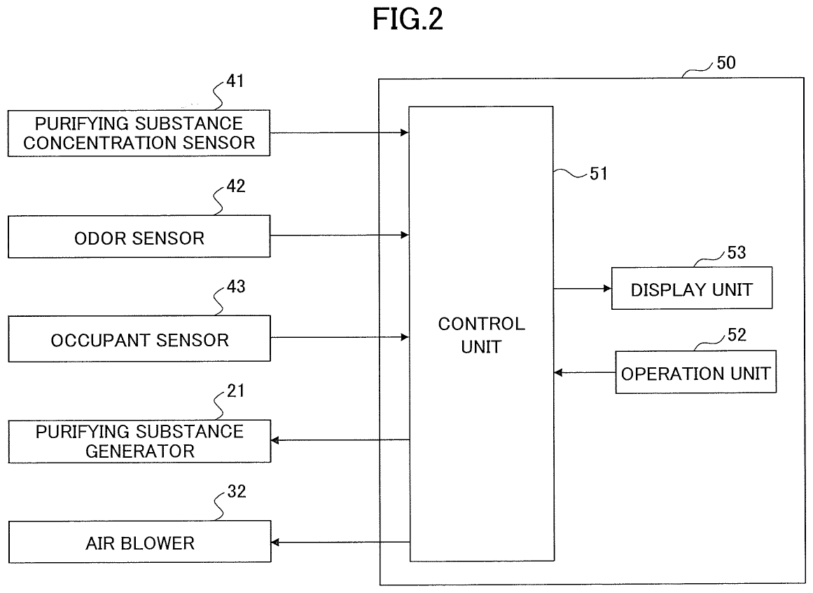

The purifying substance generator 21 uses oxygen contained in air drawn by the air blower 32, which will be described later, as a raw material, to generate ozone in the. air. The purifying substance generator 21 functions as an ozone generator. The purifying substance generator 21 is electrically connected to a control unit 51 (see FIG. 2), which will be described later, of the controller 50, and is controlled by the control unit 51 (see FIG. 2) of the controller 50. The purifying substance generator 21 may be any device that can generate ozone. For example, as the purifying substance generator 21, a discharge-type device may be used in which a discharge electrode and a counter electrode are disposed facing each other. In such a discharge-type device, an electric discharge occurs between the electrodes when voltage is applied to the electrodes. By causing air to pass between the electrodes where the electric discharge occurs, oxygen contained in the air is activated, and some dissociated or excited oxygen is converted into ozone (O₃). Accordingly, ozone is generated in the air. The generated ozone is delivered together with the air. The ozone is subjected to oxidation reaction with odor components contained in the air, thereby causing the odor components to decompose and to be removed from the air. By adjusting the amount of electric discharge, the control unit 51 (see FIG. 2) of the controller 50 can control (increase/decrease) the amount of ozone generated by the purifying substance generator 21, and control (start/stop) the generation of ozone by the purifying substance generator 21.

The dust filter 22 is disposed on the downstream side of the main body 11 relative to the purifying substance generator 21 in the air flow direction. The dust filter 22 collects solids such as dust contained in the air. The dust filter 22 may be any device that can collect dust or the like. For example, a pleated nonwoven fabric made of fine metal wires, natural fibers, or synthetic fibers may be used.

### (Circulator)

The circulator 30 includes an intake port 31, the air blower 32, and an exhaust port 33.

The intake port 31 draws the air in the space S into the main body 11. The intake port 31 is formed with slits on the side of the main body 11. Note that the intake port 31 may be formed on the top or the bottom of the main body 11.

The air blower 32 is provided in the vicinity of the intake port 31 and on the upstream side of the main body 11 relative to the purifying substance generator 21 in the air flow direction. The air blower 32 is electrically connected to the control unit 51 (see FIG. 2), which will be described later, of the controller 50, and is controlled by the control unit 51 (see FIG. 2) of the controller 50. For example, the air blower 32 may be an axial flow fan that causes air to flow in the rotational axis direction by propeller-shaped blades. The air blower 32 includes a motor (not illustrated) and a plurality of blades fixed to a rotating shaft of the motor. The air blower 32 is supplied with power from an external power source, and is driven by the motor rotating the blades. The air blower 32 draws air into the interior space of the main body 11, causes the drawn air to pass through the purifying substance generator 21 and the dust filter 22 in this order, and discharges the air from the exhaust port 33 into the space S. The rotational speed of the air blower 32 is controlled by the control unit 51 (see FIG. 2), which will be described later.

The exhaust port 33 discharges air, purified by the purifier 20 of the main body 11, into the space S of the vehicle. The exhaust port 33 is formed with slits on the side of the main body 11. Note that the exhaust port 33 may be formed on the top or the bottom of the main body 11.

### (Sensor)

The sensor 40 includes a purifying substance concentration sensor (purifying substance concentration measuring device) 41, an odor sensor (odor measuring device) 42, and an occupant sensor (occupant sensing device) 43. The purifying substance concentration sensor 41, the odor sensor 42, and the occupant sensor 43 are disposed within the space S. Further, the sensor 40 is electrically connected to the control unit 51 (see FIG. 2), which will be described later, of the controller 50, and transmits respective detection signals of the purifying substance concentration sensor 41, the odor sensor 42, and the occupant sensor 43 to the control unit 51 (see FIG. 2).

The purifying substance concentration sensor 41 measures the concentration of ozone in air in the space S, and functions as an ozone concentration sensor. The purifying substance concentration sensor 41 may be a semiconductor sensor that uses a semiconductor device. In the semiconductor sensor, the resistance of the semiconductor device changes when ozone is adsorbed on the surface of the semiconductor device, and an ozone concentration is measured based on changes in resistance.

The purifying substance concentration sensor 41 is preferably disposed near the intake port 31 rather than the exhaust port 33 in the space S. In this way, the purifying substance concentration sensor 41 can readily measure the concentration of ozone that has reacted with odor components after discharged from the exhaust port 33. Accordingly, the concentration of ozone contained in the air of the space S can be measured stably. Note that the purifying substance concentration sensor 41 may also be disposed away from the main body 11 in the space S.

The odor sensor 42 measures the concentration of odor components in the space S. The odor sensor 42 may be a semiconductor sensor that uses a metal-oxide-semiconductor such as tin oxide or zinc oxide. In the semiconductor sensor, the resistance of the semiconductor device changes when oxygen adsorbed on the surface of the semiconductor device reacts with odor components (surface reaction) and is desorbed. The concentration of odor components in the air is measured based on the change in resistance

The occupant sensor 43 detects whether there is a human present in the space S. The occupant sensor 43 may be an infrared sensor that senses body heat of a human or an animal, such as a pet, by infrared rays. In the present embodiment, the occupant sensor 43 is preferably used as the human sensing device; however, any sensor that can detect the presence of a human or an animal may be employed. For example, a sensor that uses a camera to detect motion may be employed.

### (Controller)

As illustrated in FIG. 2, the controller 50 includes the control unit 51, which is the main body, an operation unit 52, and a display unit 53. The operation unit 52 and the display unit 53 are connected to the control unit 51. In the present embodiment, the control unit 51 is provided outside the space S and inside the vehicle 12. The operation unit 52 and the display unit 53 may be provided inside the space S.

The control unit 51 controllably connects the elements constituting the air purifier 10, such as the purifying substance generator 21 and the air blower 32, to the display unit 53. The control unit 51 includes a storage device that stores a control program and various types of storage information, and an arithmetic device that is operated in accordance with the control program. The control unit 51 is implemented by the arithmetic device reading and executing the control program stored in the storage device.

The control unit 51 receives measurement results from the sensor 40. The control unit 51 controls the amount of ozone generated by the purifying substance generator 21 and the rotational speed of the air blower 32, based on the measurement results received from the sensor 40. Specifically, the control unit 51 receives a signal indicating the concentration of ozone in the space S measured by the purifying substance concentration sensor 41, a signal indicating the concentration of odor components in the space S measured by the odor sensor 42, and a signal indicating as to whether a human is detected by the occupant sensor 43. Based on the received signals, the control unit 51 calculates the concentration of ozone in the air or the concentration of odor components in the air, determines the presence or absence of a human, and outputs a signal for rotating the air blower 32 to the air blower 32 or outputs a signal for generating ozone to the purifying substance generator 21. The control unit 51 controls the amount of air blown and the amount of ozone generated by adjusting the rotational speed of the air blower 32 and the amount of electric discharge from the purifying substance generator 21.

The control unit 51 may preliminarily store, in the storage device, the relationship between the amount of ozone generated by the purifying substance generator 21 and the volume of the space S, versus the predetermined rate of increase in the concentration of ozone per unit time, namely the amount of increase in the concentration of ozone per unit time. In this case, the control unit 51 can determine whether the concentration of ozone in the space S per unit time is increasing at the predetermined rate, by comparing actual values measured by the purifying substance concentration sensor 41 with values stored in the storage unit.

The control unit 51 may control the purifying substance generator 21 and the air blower 32, such that the air purifier 10 is in one of three operation modes: ventilation mode, normal purification mode, and ozone fumigation mode.

The ventilation mode is an operation mode in which the purifying substance generator 21 does not generate ozone, and air drawn into the main body 11 is discharged from the main body 11 into the space S. In the ventilation mode, the control unit 51 performs control such that the operation of the purifying substance generator 21 is stopped and only the air blower 32 is in operation.

The normal purification mode is an operation mode in which the purifying substance generator 21 generates ozone at low concentrations, and air is purified with the low concentrations of ozone by the purifier 20 and discharged from the main body 11 into the space S together with the low concentrations of ozone. In the normal purification mode, the control unit 51 performs control such that the purifying substance generator 21 generates ozone at low concentrations and the air blower 32 is in operation.

The ozone fumigation mode is an operation mode in which the purifying substance generator 21 generates ozone at high concentrations, and air is purified with the high concentrations of ozone by the purifier 20 and discharged from the main body 11 into the space S together with the high concentrations of ozone. In the ozone fumigation mode, the control unit 51 performs control such that the purifying substance generator 21 generates ozone at high concentrations and the air blower 32 is in operation.

The concentration of ozone generated by the purifying substance generator 21 is determined based on the ratio of the amount of ozone generated by the purifying substance generator 21 to the amount of air passing through the purifying substance generator 21 (the concentration of ozone =the amount of ozone generated/the amount of air passing through the purifying substance generator 21).

Even if a constant amount of ozone is generated, the concentration of ozone discharged from the main body 11 into the space S varies as the amount of air varies. In order to increase or decrease the concentration of ozone, any of the following first to third methods can be used. The first method is a method for varying the amount of ozone generated by the purifying substance generator 21 while keeping the amount of air blown by the air blower 32 constant. The second method is a method for keeping the amount of ozone generated by the purifying substance generator 21 constant while varying the amount of air blown by the air blower 32. The third method is a method for varying both the amount of ozone generated by the purifying substance generator 21 and the amount of air blown by the air blower 32. In the present embodiment, it is preferable to employ the above-described first method to facilitate stable air circulation in the space S of the vehicle and also simplify the control of the process.

The “low concentration” refers to an ozone concentration that is less than or equal to a safety criterion value defined for human bodies, and is 0.1 ppm or less, and preferably 0.05 ppm or less.

The “high concentration” refers to an ozone concentration that exceeds the safety criterion value defined for human bodies. In order to efficiently decompose odor components, the high concentration is preferable. However, there are concerns that the high concentration may adversely affect human bodies. Therefore, the ozone fumigation mode, in which ozone is generated at high concentrations, is preferably performed after ensuring that there is no human or pet in the space S of the vehicle with the occupant sensor 43.

The operation unit 52 is provided within the space S. The operation unit 52 functions to transmit a signal for controlling the generation of ozone and the amount of ozone generated by the purifying substance generator 21 and a signal for controlling the operation and the rotational speed of the air blower 32, to the control unit 51 via external operations.

The display unit 53 is provided within the space S. The display unit 53 displays whether ozone is generated by the purifying substance generator 21 and whether the air blower 32 is operated by the air blower 32, and also displays the amount of the ozone and the rotational speed of the air blower 32. In addition, the display unit 53 displays the concentration of ozone in the space S measured by the purifying substance concentration sensor 41, the concentration of odor components in the space S measured by the odor sensor 42, and the presence or absence of a human indicated by the occupant sensor 43. Further, the display unit 53 displays the completion of an air purification method, which will be described later.

### (Operation)

The operation of the air purifier 10 having the above configuration will be described. In the air purifier 10, when an operator turns on a power source (not illustrated) of the controller 50, electrical power is supplied to the control unit 51, and the operation of the air purifier 10 is started. The operator selects the operation mode from among the ventilation mode, the normal purification mode, and the ozone fumigation mode by operating the operation unit 52. First, the ventilation mode will be described.

### (Ventilation Mode)

When the operator sets the operation mode to the ventilation mode by operating the operation unit 52, the operation unit 52 transmits a signal for setting the operation mode to the ventilation mode, to the control unit 51. In response to receiving the signal from the operation unit 52, the control unit 51 outputs a signal for driving the air blower 32 to the air blower 32, thus causing the blades of the air blower 32 to rotate. As a result, air in the space S is drawn from the intake port 31 into the main body 11. The air drawn into the main body 11 passes through the purifying substance generator 21, and enters the dust filter 22. The dust filter 22 removes solids such as dust from the air. The air from which solids have been removed may be referred to as purified air. After the purified air passes through the dust filter 22, the purified air is blown by the air blower 32 to be discharged from the exhaust port 33 into the space S. The purified air discharged from the exhaust port 33 into the space S diffuses in the space S, and is mixed with air containing odor components. The air containing the odor components circulates by natural convection in the space S. Part of the air circulating in the space S is drawn into the main body 11 from the intake port 31, and is purified again by the air purifier 10. The air purifier 10 repeatedly purify the air in the space S. Therefore, the concentration of solids, such as dust, contained in the air decreases.

### (Normal Purification Mode)

Next, an example in which the operation mode is set to the normal purification mode will be described. When the operator sets the operation mode to the normal purification mode by operating the operation unit 52, the operation unit 52 transmits a signal for setting the operation mode to the normal purification mode, to the control unit 51. In response to receiving the signal from the operation unit 52, the control unit 51 outputs a signal for driving the air blower 32 to the air blower 32, thus causing the blades of the air blower 32 to rotate. In addition, the control unit 51 outputs a signal for generating ozone at low concentrations to the purifying substance generator 21, thus causes the purifying substance generator 21 to be operated. When air drawn into the main body 11 from the intake port 31 reaches the purifying substance generator 21, the air is mixed with the low concentrations of ozone while passing through the purifying substance generator 21. The dust filter 22 removes solids such as dust from the air containing the low concentrations of ozone, which has passed through the purifying substance generator 21. The air is blown by the air blower 32 to be discharged from the exhaust port 33 into the space S, and diffuses in the space S.

While the air containing ozone diffuses in the space S, odor components contained in the air decompose and are removed by oxidation reaction with the ozone. As a result, the air is deodorized and sterilized. In addition, not only the odor components contained in the air, but also odor components adhering to the seats or the walls of the vehicle are deodorized and sterilized by oxidation reaction upon contact with the ozone. The ozone mixed with the air by the purifying substance generator 21 disappears by the reaction with the odor components contained in the air.

Note that, in addition to air from which solids have been removed, air deodorized and sterilized by reaction with ozone is also referred to as purified air. The purified air may be air that contains no odor components, or may be air that contains a trace amount of odor components.

The purified air discharged from the exhaust port 33 into the space S diffuses in the space S, and is mixed with air containing odor components. The air containing odor components circulates by natural convection in the space S. Part of the air circulating in the space S is drawn into the main body 11, and is purified again by the air purifier 10. The air purifier 10 repeatedly purifies the air in the space S, thereby allowing the air in the space S to be deodorized and sterilized. In addition, the concentration of solids contained in the air becomes low.

The concentration of the ozone and the concentration of the odor components in the air of the space S are measured by the purifying substance concentration sensor 41 and the odor sensor 42, respectively, and measurement results are transmitted to the control unit 51. Further, the occupant sensor 43 detects whether there is a human in the vehicle 12, and the detection result is transmitted to the control unit 51. Based on the above-described results, the control unit 51 outputs a signal for controlling the purifying substance generator 21 and a signal for controlling the air blower 32.

### (Ozone Fumigation Mode)

Next, an example in which the operation mode is set to the ozone fumigation mode will be described. The ozone fumigation mode is the same as the normal purification mode, except that the control unit 51 outputs a signal for generating ozone at high concentrations to the purifying substance generator 21, such that the purifying substance generator 21 generates ozone at high concentrations.

In the ozone fumigation mode, while odor components are contained in air of the space S, ozone is consumed by reaction with the odor components. However, the purifying substance generator 21 introduces additional high concentrations of ozone into air that has been drawn into the main body 11. Therefore, the concentration of ozone in the air of the space S repeatedly increase or decrease and is unstable.

Upon the removal of all odor components that react with ozone from the air, the concentration of the ozone in the air of the space S increases at a predetermined rate. Note that ozone naturally decomposes and disappears in the process of being discharged into the space S together with air from the purifying substance generator 21. However, the rate at which ozone naturally decomposes is significantly lower than the rate at which ozone decomposes by reaction with odor components and lower than the rate at which additional ozone is supplied by the purifying substance generator 21. Thus, the decomposition rate of ozone is not required to be taken into account to determine an increase or a decrease in the concentration of ozone.

Further, the ozone fumigation mode uses high concentrations of ozone that may adversely affect human bodies. Thus, it is preferable to use the occupant sensor 43 to ensure that there are no humans or pets in the space S before starting operation. In addition, it is desirable to inform the user that the user can enter the vehicle once the concentration of ozone reaches or drops below a safe level after the generation of ozone is stopped, or to unlock the door lock after the concentration of ozone reaches or drops below the safe level.

### <Air Purification Method>

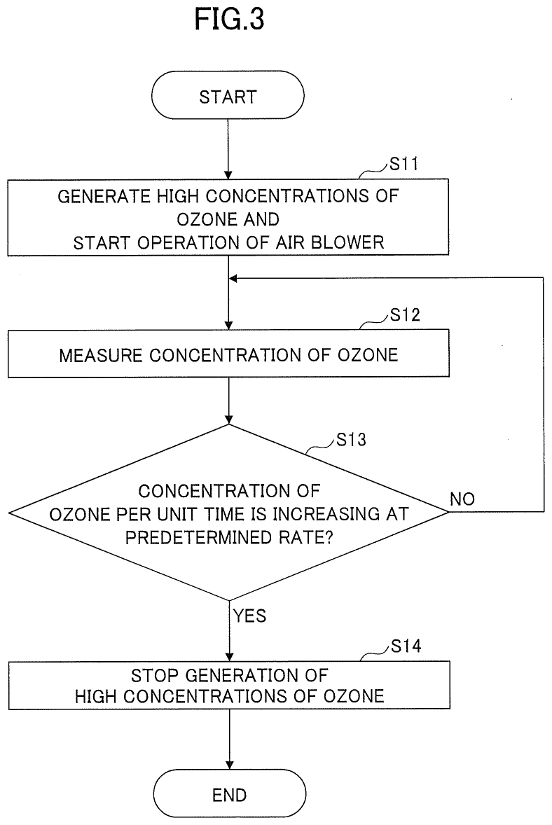

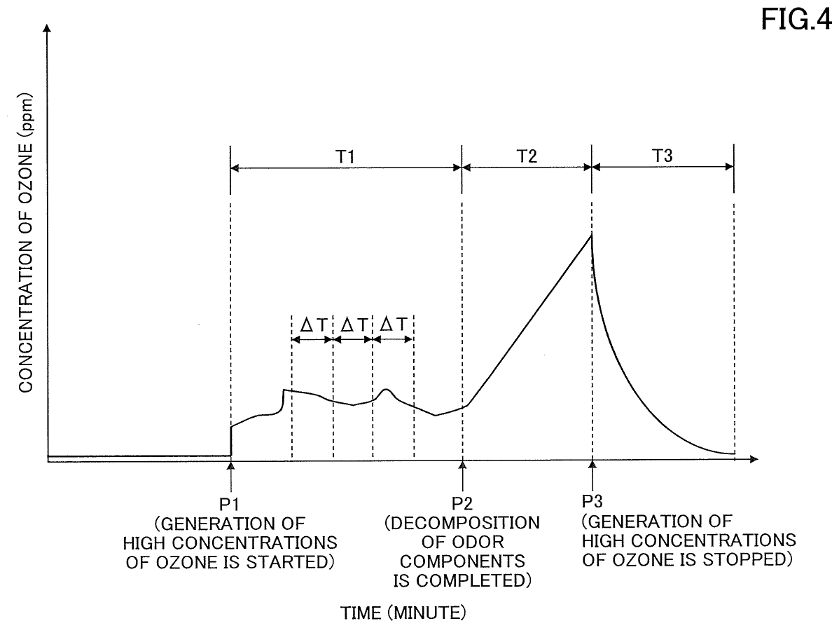

Next, an example of an air purification method performed by the air purifier 10 having the above-described configuration will be described with reference to FIG. 3 and FIG. 4.

FIG. 3 is a flowchart illustrating the air purification method. FIG. 4 is a graph indicating the relationship between the time and the concentration of ozone. As illustrated in FIG. 3, the control unit 51 selects the ozone fumigation mode as the operation mode, and starts the operation of the purifying substance generator 21 and the operation of the air blower 32 (step S11). Accordingly, the air blower 32 causes air in the space S to be drawn into the main body 11, and the purifying substance generator 21 generates high concentrations of ozone. The generated high concentrations of ozone allow solids to be removed from air, and the purified (deodorized and sterilized) air is discharged from the exhaust port 33 of the air purifier 10 and diffuses in the space S. Therefore, as illustrated in FIG. 4, at time point P1, the concentration of ozone in the air of the space S increases, and odor components in the space S reacts with the ozone and decomposes.

Next, the purifying substance concentration sensor 41 measures the concentration of ozone in the space S, and transmits a measurement result to the control unit 51 (step S12).

The control unit 51 determines whether the concentration of ozone per unit time, measured by the purifying substance concentration sensor 41, is increasing at a predetermined rate (step S13).

In the present embodiment, the “predetermined rate” means that the previously-measured amount of increase in the concentration of ozone per unit time is the same as or approximately the same as the current amount of increase in the concentration of ozone per unit time. The . “approximately the same” means that an error of several percent is allowed.

As a method for calculating the concentration of ozone per unit time, the value of an ozone concentration after unit time ΔT, the average value of the sum of ozone concentrations in unit time ΔT, or the value of an ozone concentration at a middle time of unit time ΔT may be calculated.

In step S13, when it is determined that the concentration of ozone per unit time is not increasing at the predetermined rate, the control unit 51 continues the operation of the purifying substance generator 21. Then, the control unit 51 causes the purifying substance concentration sensor 41 to measure the concentration of ozone in the space S (step S12), and determines whether the concentration of ozone per unit time is increasing at the predetermined rate (step S13). The concentration of ozone does not continuously increase for a predetermined period of time after the operation of the purifying substance generator 21 is started, because ozone is consumed by reaction with odor components. For example, as illustrated in FIG. 4, for a period of time T1, while ozone is consumed by reaction with odor components, additional ozone is introduced into air by the purifying substance generator 21. Therefore, the concentration of ozone in the space S varies and is not stable, and the concentration of ozone per unit time ΔT does not tend to increase. The period of time T1 and the degree of changes in the concentration of ozone depend on the concentration of odor components in the space S of the vehicle.

In step S13, when it is determined that the concentration of ozone per unit time is increasing at the predetermined rate, the control unit 51 causes the purifying substance generator 21 to stop the generation of high concentrations of ozone (step S14). When the predetermined period of time elapses from the start of the generation of ozone, and approximately all odor components in the space S decompose, the decomposition of odor components is completed. As a result, because there are approximately no substances that react with ozone in the space S, the concentration of ozone tends to continuously increase. For example, as illustrated in FIG. 4, there are approximately no substances that react with ozone in the space S at time point P2. Accordingly, for a period of time T2, the concentration of ozone in the space S continuously increases, and the concentration of ozone per unit time increases at the predetermined rate.

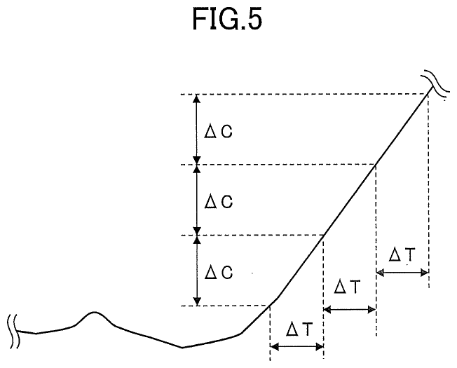

As an indicator for determining whether the concentration of ozone per unit time is increasing at the predetermined rate, the amount of increase ΔC in the concentration of ozone per unit time ΔT may be used as illustrated in FIG. 5. For example, when the amount of increase ΔC in the concentration of ozone per unit time ΔT is measured several consecutive times (three times or more, for example) and the measured values of the amount of increase ΔC are approximately the same, it can be determined that the concentration of ozone per unit time is increasing at the predetermined rate.

The predetermined rate of increase in the concentration of ozone per unit time, namely the amount of increase in the concentration of ozone per unit time is preferably constant. When the amount of increase in the concentration of ozone is constant, the control unit 51 can readily determine that the concentration of ozone per unit time is increasing at the predetermined rate. Accordingly, it can be more securely determined that all odor components in the space S have been removed.

When the control unit 51 determines that the concentration of ozone per unit time, measured by the purifying substance concentration sensor 41, is increasing at the predetermined rate, the control unit 51 causes the purifying substance generator 21 to stop the generation of high concentrations of ozone. In the present embodiment, the control unit 51 stops the operation of the purifying substance generator 21, such that the purifying substance generator 21 does not generate ozone at high concentrations and the concentration of ozone in the space S is thus gradually decreased. However, instead of stopping the operation of the purifying substance generator 21, the control unit 51 may perform control such that the generation of high concentrations of ozone is switched to the generation of low concentrations of ozone, or may perform control such that the concentration of ozone gradually decreases.

Further, it is preferable for the control unit 51 to forcibly stop the operation of the purifying substance generator 21 when the concentration of ozone in the space S reaches a predetermined value. Accordingly, it is possible to reduce the amount of ozone excessively generated in the space S.

When the operation of the purifying substance generator 21 is stopped, the supply of ozone from the purifying substance generator 21 into the space S is stopped. That is, no additional ozone is supplied into the space S. Because ozone in the space S naturally decomposes, the concentration of ozone in the space S decreases for a period of time T3 after the operation of the purifying substance generator 21 is stopped at time point P3, as illustrated in FIG. 4.

The above-described air purifier 10 includes the control unit 51. When the control unit 51 determines that the concentration of ozone per unit time is increasing at the predetermined rate, the control unit 51 adjust the amount of ozone by controlling the purifying substance generator 21. A constant amount of ozone is generated by the purifying substance generator 21 and is supplied into the space S. When all odor components in the space S decompose by reaction with ozone, there are approximately no substances that react with ozone in the space S, and thus, the concentration of ozone in the space S increases. Therefore, it can be said that there are no odor components in the space S when the concentration of ozone per unit time is increasing at the predetermined rate. In the air purifier 10, the control unit 51 stops the operation of the purifying substance generator 21 when the concentration of ozone in the space per unit time is increasing at the predetermined rate. Accordingly, the air purifier 10 can stop the generation of ozone at an appropriate timing, and adjust the amount of ozone generated appropriately.

In addition, because the air purifier 10 can stop the generation ozone at an appropriate timing, it is possible to reduce the generation of excess ozone. Accordingly, the air purifier 10 can efficiently remove odor components while reducing wasteful energy consumption.

Note that, in the present embodiment, the odor sensor 42 provided in the space S may be a semiconductor-based sensor or many be any other sensor.

In the present embodiment, the storage device preliminarily stores the relationship between the amount of ozone generated by the purifying substance generator 21 and the volume of the space S, versus the predetermined rate of increase in the concentration of ozone per unit time, namely the amount of increase in the concentration of ozone per unit time. Accordingly, the control unit 51 can readily determine whether the concentration of ozone in the space S is increasing at the predetermined rate, by comparing values measured by the purifying substance concentration sensor 41 with values stored in the storage device. Therefore, it is possible for the control unit 51 to readily control the purifying substance generator 21.

In the present embodiment, the control unit 51 can forcibly stop the operation of the purifying substance generator 21 when the concentration of ozone in the space S reaches the predetermined value. Accordingly, it is possible to reduce the amount of ozone excessively generated in the space S.

In the present embodiment, the control unit 51 sets the operation mode to one of the ventilation mode, the normal purification mode, and the ozone fumigation mode. Accordingly, the air purifier 10 can appropriately purify air in accordance with the concentration of odor components in the space S.

In the above embodiment, an example in which the air purifier 10 purifies air in the space S of the vehicle 12 has been described. However, the air purifier 10 can purify air in any space at an appropriate timing as described above. For example, the air purifier 10 may be appropriately installed in a space of a train, an airplane, or a building, and used to purify air in the space. Further, the air purifier 10 may be installed in a kitchen of a restaurant, a partitioned smoking area of a public facility, a space of a building at a garbage collection station, an interior storage space of an air conditioner or a refrigerator, or an air flow path for deodorization.

In the present embodiment, the main body 11 that includes the purifier 20 and the circulator 30 is provided inside the space S of the vehicle 12; however, the present invention is not limited thereto. For example, the main body 11 may be provided outside the space S and inside the vehicle 12, and the intake port 31 and the exhaust port 33 of the circulator 30 may be open toward the space S.

In the present embodiment, the purifying substance generator 21 generates ozone as a substance that purifies odor components; however, any substance that can remove odor components from air may be used. For example, the purifying substance generator 21 may generate an oxidizer such as hypochlorous acid (HClO) or hydrogen peroxide (H₂O₂).

When the purifying substance generator 21 generates hypochlorous acid as a purifying substance, the purifying substance concentration sensor 41 uses a hypochlorous acid concentration sensor that can measure the concentration of hypochlorous acid in air in the space S. For example, the purifying substance concentration sensor 41 may use a measurement device that includes a fluorescence reagent that emits light upon reaction with hypochlorous acid generated by the purifying substance generator 21. The fluorescence reagent of the purifying substance concentration sensor 41 emits lights upon contact with hypochlorous acid. The purifying substance concentration sensor 41 can determine the presence or absence of hypochlorous acid and measure the concentration of hypochlorous acid based on light emission from the fluorescence reagent and the light intensity.

In the present embodiment, the purifying substance generator 21 and the air blower 32 are included in the main body 11; however, the purifying substance generator 21 and the air blower 32 are not necessarily included in the main body 11.

In the present embodiment, the air blower 32 is provided on the upstream side of the main body 11 relative to the purifying substance generator 21 in the air flow direction.

In the present embodiment, the purifier 20 includes the dust filter 22; however, the purifier 20 is not required to include the dust filter 22 if there is no need to collect solids such as dust contained in air.

In the present embodiment, the dust filter 22 is used to collect solids such as dust contained in air; however any device that can remove solids such as dust from air can be used. For example, an electrostatic precipitator may be used instead of the dust filter 22. The electrostatic precipitator removes dust particles from air by passing high voltage through the air, charging the dust particles, and causing the dust particles to electrically stick to a dust collector. The maintenance cycle of the electrostatic precipitator is longer than that of the dust filter 22. Thus, the frequency of maintenance can be reduced.

In the present embodiment, the control unit 51 of the controller 50 is provided outside the space S and inside the vehicle 12, and the operation unit 52 and the display unit 53 are provided inside the space S; however, the present invention is not limited thereto. The entire controller 50 may be provided in the main body 11, or only the control unit 51 of the controller 50 may be provided in the main body 11. Alternatively, the operation unit 52 and the display unit 53 of the controller 50 may be provided outside the vehicle 12, or the entire controller 50 may be provided outside the vehicle 12.

Although the specific embodiments have been described above, the present invention is not limited to the particulars of the described embodiments. The embodiments may be implemented in various other forms, and various combinations, omissions, substitutions, and modifications may be made without departing from the scope of the present invention. The embodiments and variations thereof are included in the scope and gist of the present invention and are included in the scope of the present invention described in the claims and equivalents thereto.

What is claimed is

1. An air purifier comprising:
a purifier configured to remove an odor component from air in a space, the purifier including a purifying substance generator configured to generate a purifying substance that removes the odor component;
a circulator configured to draw the air in the space into the purifier, and discharge the air purified by the purifier into the space,
a purifying substance concentration sensor configured to detect a concentration of the purifying substance in the air, and
a controller configured to control an amount of the purifying substance generated by the purifying substance generator, in accordance with the concentration of the purifying substance, wherein
the controller adjusts the amount of the purifying substance generated by the purifying substance generator by controlling the purifying substance generator, in response to determining that an increase rate in the concentration of the purifying substance per unit time is greater than a predetermined rate.

2. The air purifier according to claim 1, wherein the controller determines the increase rate in the concentration of the purifying substance per unit time, based on the amount of the purifying substance generated by the purifying substance generator and a volume of the space.

3. The air purifier according to claim 1, wherein the controller stops operation of the purifying substance generator, in response to the increase rate in the concentration of the purifying substance in the space reaching a predetermined value.

4. The air purifier according to claim 1, further comprising a main body that includes the purifier, wherein
the controller sets an operation mode to any one of:
a ventilation mode in which the purifying substance generator does not generate the purifying substance, and the air drawn into the main body is discharged from the main body into the space;
a normal purification mode in which the purifying substance generator generates the purifying substance at a low concentration, and the air is purified with the low concentration of the purifying substance by the purifier and discharged from the main body into the space together with the low concentration of the purifying substance; and
a fumigation mode in which the purifying substance generator generates the purifying substance at a high concentration, and the air is purified with the high concentration of the purifying substance by the purifier and discharged from the main body into the space together with the high concentration of the purifying substance.

5. The air purifier according to claim 1, further comprising an odor sensor configured to measure a concentration of the odor component in the space.

6. The air purifier according to claim 1, further comprising an occupant sensor configured to detect whether there is a human in the space.

7. The air purifier according to claim 1, wherein the purifying substance is ozone or hypochlorous acid.

8. The air purifier according to claim 7, wherein, in a case where the purifying substance is the hypochlorous acid, the purifying substance concentration sensor uses a fluorescence reagent that emits light upon reaction with the hypochlorous acid to calculate a hypochlorous acid concentration.

9. An air purification method comprising:
generating a purifying substance in a space;
removing an odor component from air in the space;
detecting a concentration of the purifying substance in the space; and
adjusting an amount of the purifying substance generated in the space, in response to determining that an increase rate in the concentration of the purifying substance per unit time is greater than a predetermined rate.

10. The air purification method according to claim 9, wherein the purifying substance is ozone or hypochlorous acid.
